(19) United States
(12) Patent Application Publication
(10) Pub. No.: US 2021/0322615 A1
(43) Pub. Date: Oct. 21, 2021

(54) Walk-Through Sanitizing Nebulization System

(71) Applicant: X-VIRUS LLC, Las Vegas, NV (US)

(72) Inventors: Carlos Ramos, Bonita, CA (US); Martin Simon, Woodland Hills, CA (US); Fernando Flores, Benito Juarez (MX)

(21) Appl. No.: 17/235,865

(22) Filed: Apr. 20, 2021

Related U.S. Application Data
(60) Provisional application No. 63/012,913, filed on Apr. 20, 2020.
(60) Provisional application No. 63/029,329, filed on May 22, 2020.
(60) Provisional application No. 63/033,181, filed on Jun. 1, 2020.
(60) Provisional application No. 63/057,805, filed on Jul. 28, 2020.
(60) Provisional application No. 63/058,416, filed on Jul. 29, 2020.

(51) Int. Cl. A61L 2/26; A61L 2/16

(52) U.S. Cl. CPC A61L 2/26; A61L 2/16; A61L 2202/20; A61L 2202/14; A61L 2202/15

(57) ABSTRACT

Walk-through sanitizing nebulization system configured to permit people to walk through the system and are briefly, but sufficiently, exposed to a nebulized sanitizer for eliminating bacteria and viruses that may be present on the exterior surface of a person including skin and clothing.

### RELATED APPLICATIONS

This application claims the benefit of U.S. provisional application No. 63/012,913, filed Apr. 20, 2020; U.S. provisional application No. 63/029,329, filed May 22, 2020; U.S. provisional application No. 63/033,181, filed Jun. 1, 2020; U.S. provisional application No. 63/057,805, filed Jul. 28, 2020; and U.S. provisional application No. 63/058,416, filed Jul. 29, 2020, all of which are incorporated in their entirety by reference herein.

### BACKGROUND

The recent global pandemic caused by the SARS-CoV-2 virus has created an urgent need to avoid person-to-person transmission of COVID-19 disease. Precautionary measures include the avoidance of personal contact, donning of masks and gloves, social distancing, and the use of sanitizing products to reduce the spread and transmission of the COVID-19 virus. As the virus can be transmitted by human contact, SARS-CoV-2 can survive on the skin and clothing of a person. Thus, there exists an urgent need to eliminate or significantly reduce the existence of the virus and other harmful pathogens that may be present on people who are gathering in public places, especially when entering a confined area such as a building or other structure, or at an entrance to an area of mass gathering of people. This need can be met appropriately only with a sanitizing system that is safe, efficient, and quick, with minimal inconvenience and disruption to persons using it.

### SUMMARY

The present invention is directed to a sanitizing nebulization system configured to permit people to walk there through. People walking through the system are briefly, but sufficiently, exposed to a nebulized sanitizer for killing bacteria and viruses that may be present on the exterior surface of a person including skin and clothing.

As a person approaches the entrance of the sanitizing nebulization system, a thermo sensing device measures the person's body temperature and shows the temperature on a display to determine if the person has a fever. In the event the person is febrile, that person would be denied entry and directed to seek immediate medical attention for further evaluation.

If the person does not have a fever, the person can use the provided hand sanitizer to disinfect their hands located near the entrance of the system and then enter through the entrance of the sanitizing nebulization system. The person then continues to walk through the sanitizing system at a normal walking pace. Motion sensors detect the presence of the person and activate one or more fine mist spray nozzles such as nebulizers or atomizers to disperse a fine mist or fog of a disinfectant, sanitizing solution. The sanitizing solution is delivered to the nozzles preferably by a hydro pump at approximately 4 kg/cm² of pressure (or other desired suitable pressure) over high pressure hoses. The person walks through the mist or fog and exits from the opposite end of the sanitizing nebulization system. A preferred rate of sanitization is approximately 12 people per minute walking one at a time through the sanitizing nebulization system. In approximately 5 seconds, an individual can be effectively sanitized prior to enter a building, confined space, or mass gathering area.

The sanitizing solution is preferably an organic broad spectrum disinfectant that does not irritate eyes, cause skin allergies, or stain clothes. The sanitizing solution preferably disinfects clothes, hair, shoes, accessories exposed to the solution. It is appreciated that many commercially available sanitizing solutions suitable for the intended purpose may be used with the sanitizing system of the present invention.

The sanitizing nebulization system of the present invention is configured to be an open design; that is, having open sides and an open ceiling to avoid an undesirable closed tunnel effect and avoid any unwanted accumulation of the dispensed sanitizing solution within the device. Instead, the nebulized sanitizing solution is permitted to readily and easily dissipate into the surrounding environment. The spaced apart and open design avoids any direct contact by a person walking through the system for a contactless experience. The open air design is less obstructive and intrusive to the surrounding environment and helps those who experience claustrophobia.

The sanitizing nebulization system of the present invention with its high throughput capability has many applications and uses in various settings such as entrances to hospitals, pharmacies, airports, hotels, office buildings, sports arenas, amusement parts, and museums among many other public places in which rapid disinfecting of a large number of people with minimal intrusion and disruption is desired.

### BRIEF DESCRIPTION OF THE DRAWINGS

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1 is a front, top and right perspective view of an embodiment of the sanitizing nebulization system.

FIG. 2 is a right side elevation view of an embodiment of the sanitizing nebulization system.

FIG. 3 is a left side elevation view of an embodiment of the sanitizing nebulization system.

FIG. 4 is a front elevation view of an embodiment of the sanitizing nebulization system.

FIG. 5 is a rear elevation view of an embodiment of the sanitizing nebulization system.

FIG. 6 is a top plan view of an embodiment of the sanitizing nebulization system.

FIG. 7 is a bottom plan view of an embodiment of the sanitizing nebulization system.

FIGS. 8 and 9 are perspective views of an embodiment of the walk-through sanitizing nebulization system.

FIGS. 10A-10C show a side elevation, front end view, and front side perspective views, respectively, of an embodiment of the sanitizing nebulization system.

FIG. 11 is a front perspective view of the sanitizing solution container, pump, and power supply of an embodiment of the sanitizing nebulization system.

FIGS. 12A-12D, illustrate the hydraulic system of an embodiment of the sanitizing nebulization system.

FIGS. 13A-13D, illustrate the electrical system of an embodiment of the sanitizing nebulization system.

FIG. 14 is a front, top and right perspective of another embodiment of the sanitizing nebulization system.

FIG. 15 is a right side elevation view of an embodiment of the sanitizing nebulization system.

FIG. 16 is a left side elevation view of an embodiment of the sanitizing nebulization system.

FIG. 17 is a front elevation view of an embodiment of the sanitizing nebulization system.

FIG. 18 is a rear elevation view of an embodiment of the sanitizing nebulization system.

FIG. 19 is a top plan view of an embodiment of the sanitizing nebulization system.

FIG. 20 is a bottom plan view of an embodiment of the sanitizing nebulization system.

FIG. 21 is a table a material data for an embodiment of the sanitizing solution for use with an embodiment of the sanitizing nebulization system.

### DETAILED DESCRIPTION

Aspects of the present disclosure are best understood from the following detailed description when read with the accompanying Figures. It is noted that, in accordance with the standard practice in the industry, various features are not drawn to scale. In fact, the dimensions of the various features may be arbitrarily increased or reduced for clarity of discussion.

The following disclosure provides many different embodiments, or examples, for implementing different features of the provided subject matter. Specific examples of components, applications and arrangements are described below to simplify the present disclosure. These are, of course, merely examples and are not intended to be limiting. For example, the attachment of a first feature and a second feature in the description that follows may include embodiments in which the first feature and the second feature are attached in direct contact, and may also include embodiments in which additional features may be positioned between the first feature and the second feature, such that the first feature and the second feature may not be in direct contact. In addition, the present disclosure may repeat reference numerals and/or letters in the various examples. This repetition is for the purpose of simplicity and clarity and does not in itself dictate a relationship between the various embodiments and/or configurations discussed.

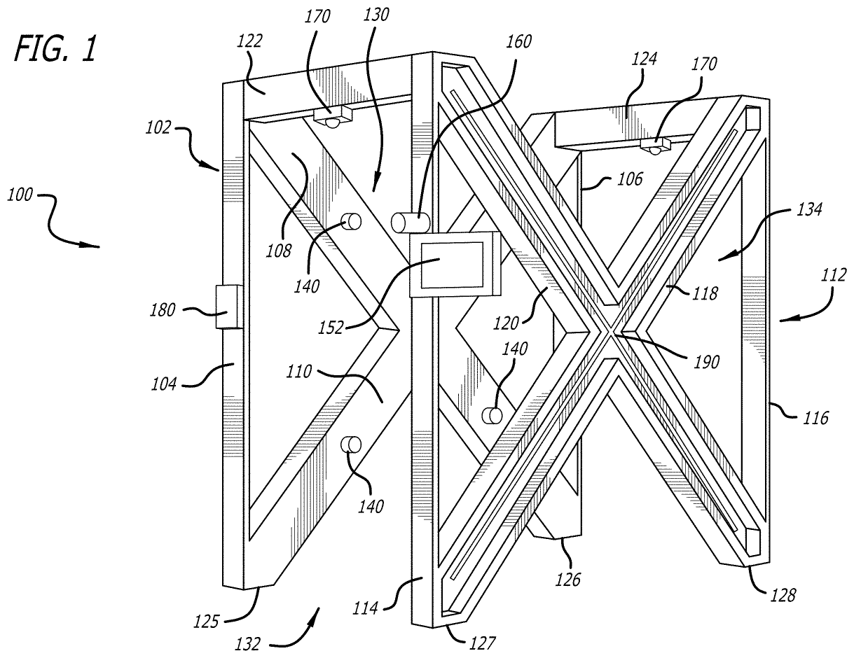

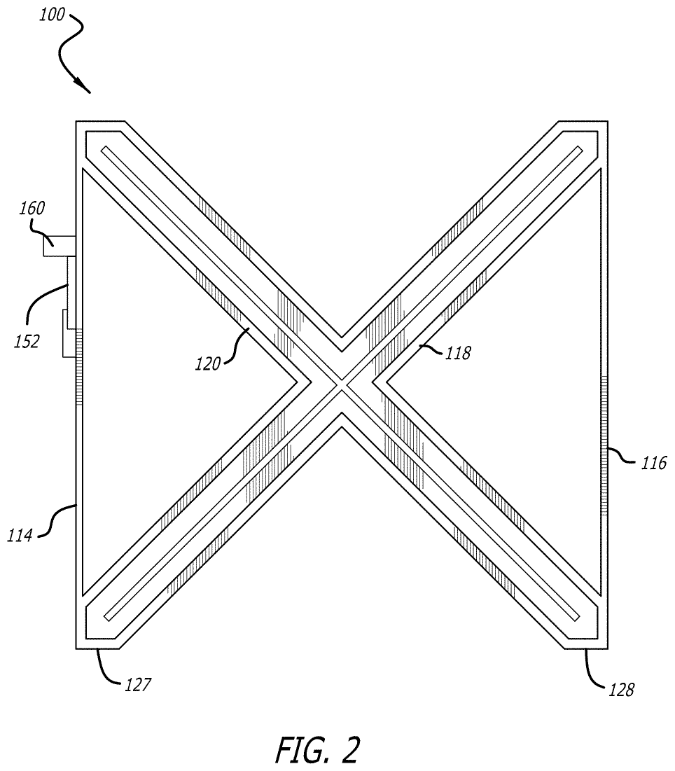

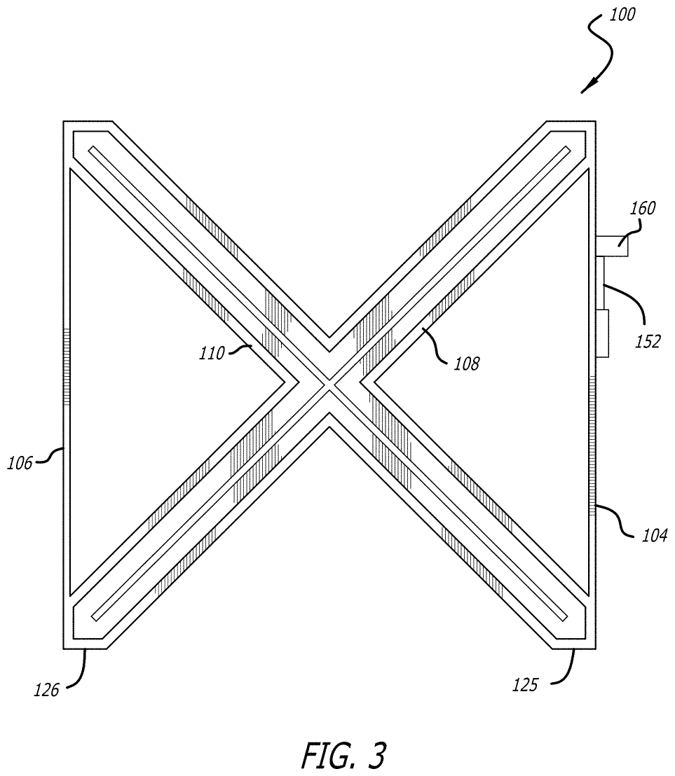

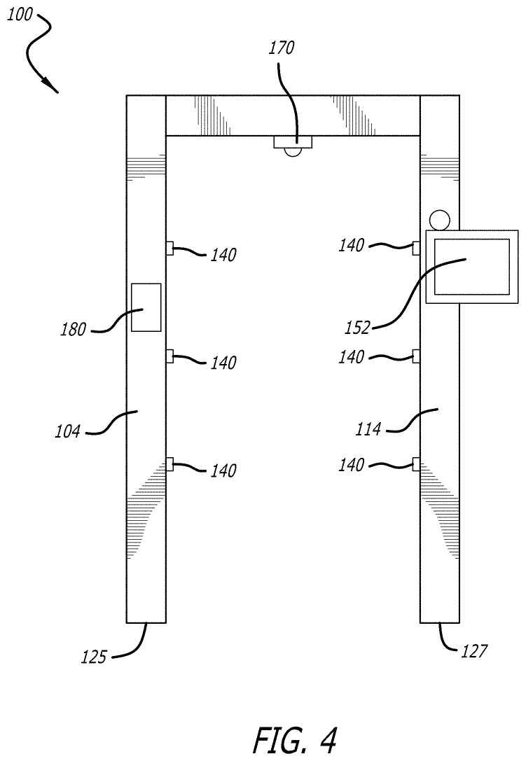

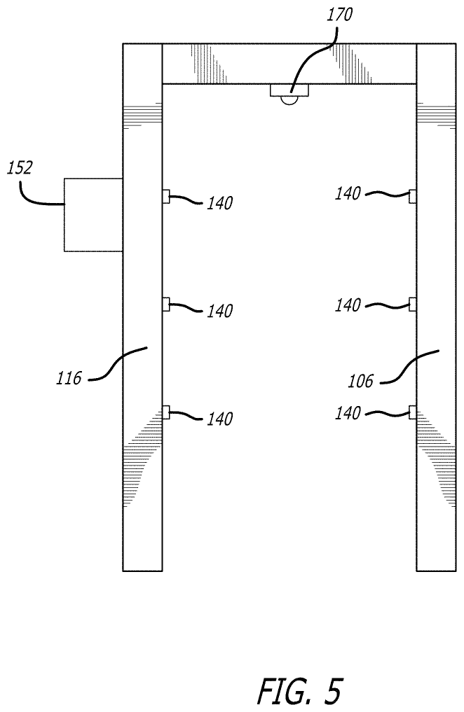

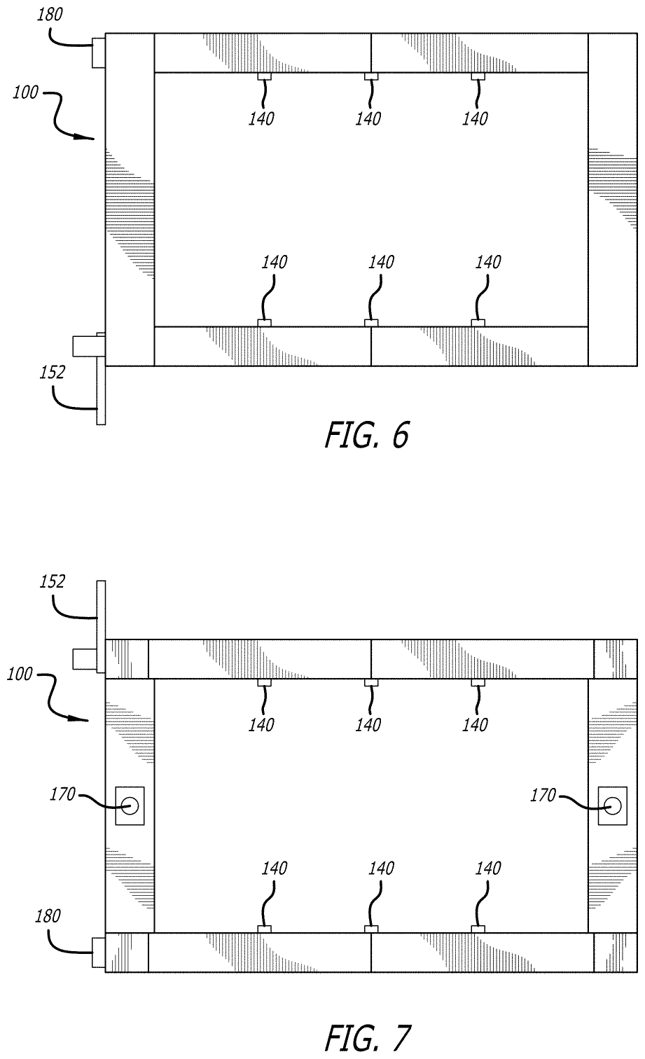

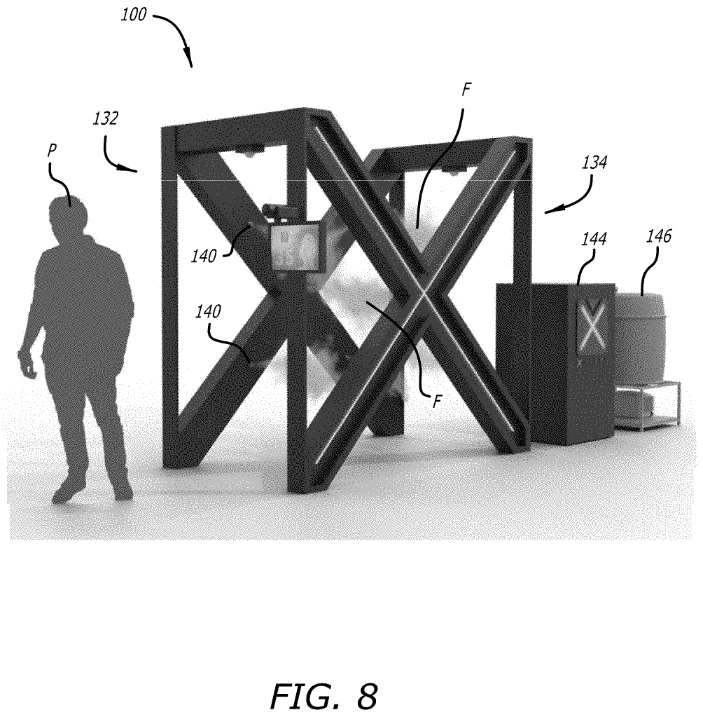

FIGS. 1-7, show an embodiment of the sanitizing nebulization system 100 having an open frame structure including a first side portion 102 and an opposite second side portion 112. First side portion 102 includes a front vertical member 104, a rear vertical member 106, a cross connector 108, and a cross connector 110. Each of cross connector 108 and cross connection 110 connect to both front vertical member 104 and rear vertical member 106, respectively, and preferably intersect to form an X-shaped configuration. In this arrangement, first side portion has a generally open configuration with large open areas between vertical members 104, 106 and cross connectors 108, 110. Near the bottom junctures of cross connectors 108, 110 and front and rear vertical members 104, 106 are preferably formed flattened areas 125 and 126 for providing stable contact surfaces for supporting first side portion 102 when placed on the ground.

Second side portion 112 has a front vertical member 114, a rear vertical member 116, a cross connector 118, and a cross connector 120. Each of cross connector 118 and cross connection 210 connect to both front vertical member 114 and rear vertical member 116, respectively, and preferably intersect to form an X-shaped configuration. In this arrangement, second side portion 112 has a generally open configuration with large open areas between vertical members 114, 116 and cross connectors 118, 120. Near the bottom junctures of cross connectors 118, 120 and front and rear vertical members 114, 116 are preferably formed flattened areas 127 and 128 for providing stable contact surfaces for supporting first side portion 112 when placed on the ground.

First side portion 102 and second side portion 112 are preferably connected by a front interconnecting member 122 and a rear interconnecting member 124. Second side portion 112 is spaced apart from and opposite to first side portion 102 to define an interior area 130 therebetween. Interior area 130 is in open communication with the exterior environment surrounding system 100 through the open ceiling and the open configuration of opposite first and second side portions 102 and 112. At one end of sanitizing nebulization system 100 is an open entrance 132 for entering interior area 130 and an open exit 134 at the opposite end provides an egress from interior area 130. Entrance 132 is defined by front vertical members 104, 114 and front interconnecting member 122. Exit 134 is defined by rear vertical members 106, 116 and rear interconnecting member 124. As entrance 132 and exit 134 are unobstructed, they provide further open access from interior area 130 to the surrounding environment.

The open configuration of sanitizing nebulization system 100 does not allow any bacteria or viruses to adhere to walls or a ceiling, because there are no walls or ceiling. The minimum surface area of the exposed surfaces can be covered with virus-repellent paint to further minimize the potential for contamination. The open and spaced apart configuration is such that a person walking though has no direct contact with any part of system 100, providing a contactless experience. The visible exterior surfaces can be painted or wrapped with advertising or company logos for branding and promotional purposes.

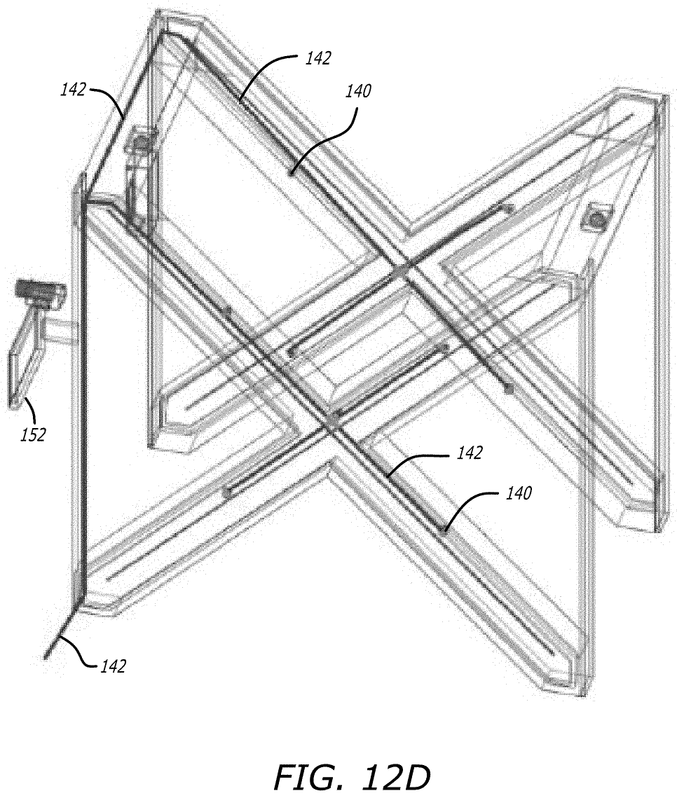

As shown in FIGS. 1, 4-7, and 12A-12D, nozzles 140 are positioned along the interior area 130 facing surfaces of cross connectors 108, 110,118, and 120. Nozzles 140 are configured to deliver a sanitizing solution as a mist or nebulized fog to the interior area 130 such that a person P walking through sanitizing nebulization system 100 would be exposed to the fogged sanitizing solution. The person walks through the mist or fog and exits from the opposite end of sanitizing nebulization system 100. An example of a preferred rate of sanitization is approximately 12 people per minute walking one at a time through the sanitization nebulization system 100. In approximately 5 seconds, an individual can be effectively sanitized prior to enter a building, confined space, or mass gathering area.

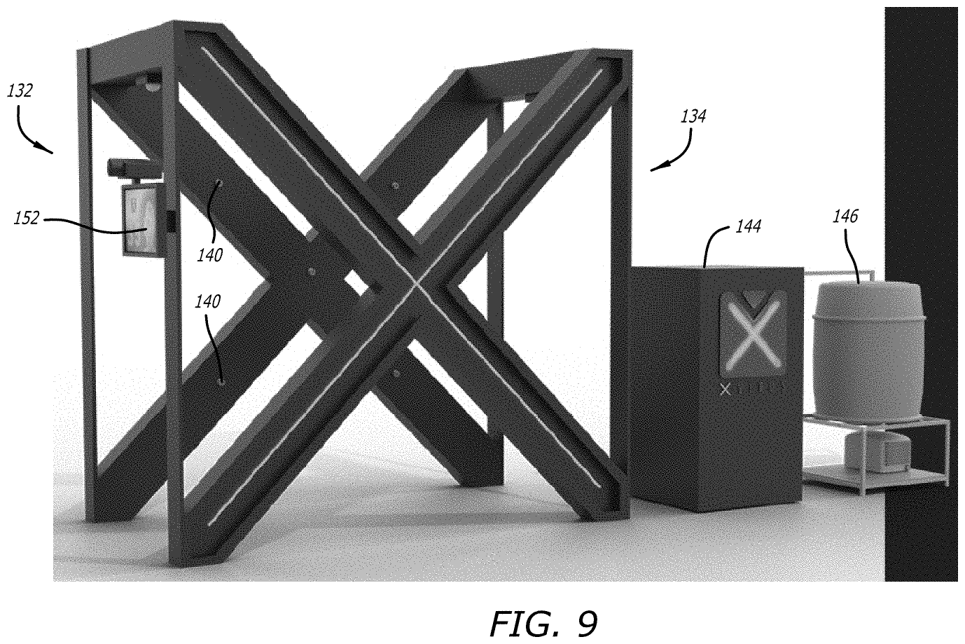

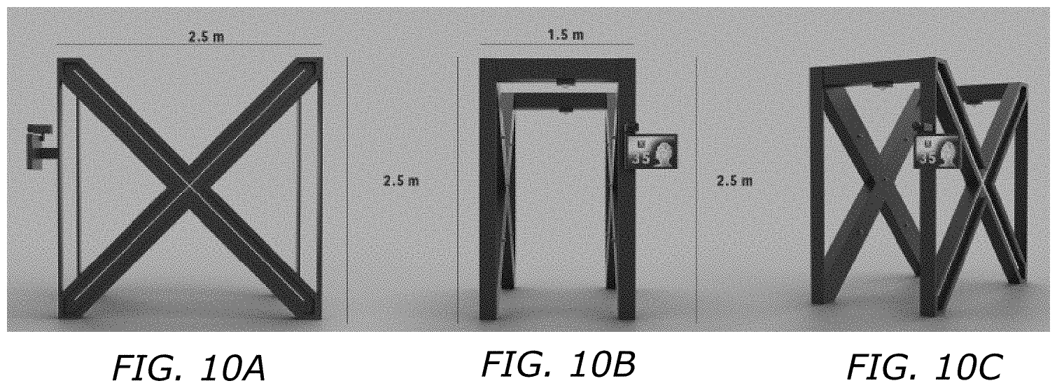

As shown in FIGS. 8, 9, and 12A-12D, the hydraulic system preferably includes tubing 142 connected to a pump 144 and in communication with a sanitizing solution container 146 for storing the sanitizing solution. Tubing 142 is preferably hidden within the framework of first and second side portions 102, 112, and interconnecting members 122, 124. The sanitizing solution is delivered to nozzles 140 by hydro pump 144 preferably at approximately 4 kg/cm² of pressure (or other desired suitable pressure) over high pressure hoses 142. Other components include the fogging system with waits 0.2 10 pieces, high pressure hose with brass connectors; 4 kilos pressure hydro pump made by Karcher mold k2; 2×1 ptr rack with 200 liter container and space for hydro motor; Acii-240v model infrared sensor. The hydraulic system preferably works with a ¼ hp Karcher model ks hydraulic pump with 4 kilos of pressure connected to 110 volts and a 200 liter container which will send the H2O and the disinfecting solution through all the high pressure ⅓ until reaching 0.2 micro nebulization waiting times (10) connected with a brass tee each.

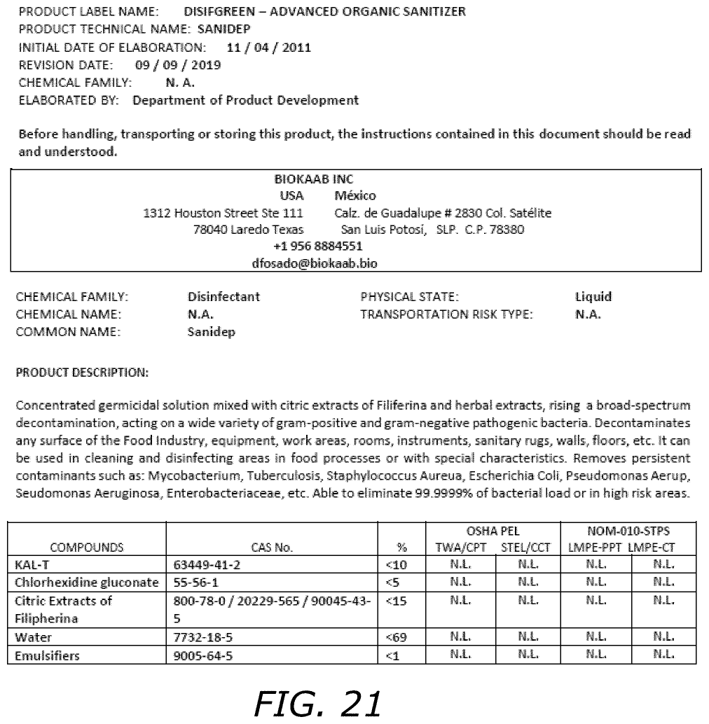

Referring to FIG. 21, the sanitizing solution is preferably an organic broad spectrum disinfectant such as for example, but not limited to, DISIFGREEN (Sanidep) by Biokaab, Inc. Laredo, Tex. In the preferred embodiment, the sanitizing solution does not irritate eyes, cause skin allergies, or stain clothes. The sanitizing solution preferably disinfects clothes, hair, shoes, accessories exposed to the solution. It is appreciated that many commercially available sanitizing solutions suitable for the intended purpose may be used with the sanitizing system of the present invention. The sanitizing solution may be stored in large quantities such as for example in a 200 liter container that could supply sufficient solution to last approximately 12 hours of operation of the sanitizing system. It is appreciated that greater or lesser quantities may be stored for use with the sanitizing system as desired.

In an embodiment, a sanitizer is used that complies with the best technology and design to combat any type of virus, bacteria, spores, and fungi with the best nebulization system. The sanitizer is preferably a 100% organic broad-spectrum disinfectant made from citrus by removing an extract from the tangerine see. The disinfectant is released through a nebulization system. It does not irritate eyes, cause skin allergies, or stain clothes, hair, shoes, or accessories. Its effective action can last up to 72 hours.

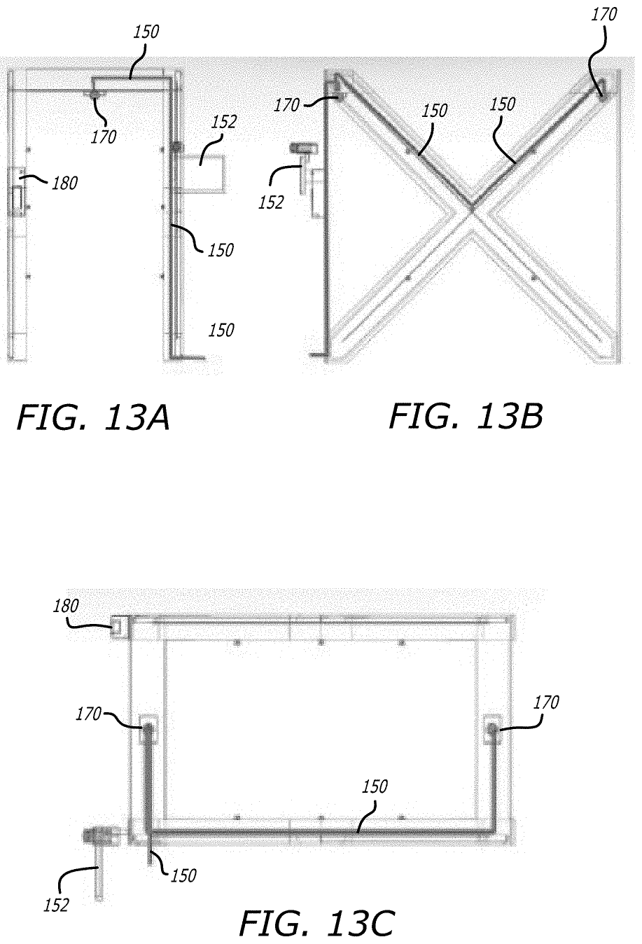

As shown in FIGS. 13A-13D, similarly, wiring 150 is preferably hidden from external view within the framework of first and second side portions 102, 112, and interconnecting members 122, 124. Wiring 150 connects to a power source to power one or more of a display 152, a computer processing unit (CPU) 154, a thermo sensor 160, a motion sensor 170, and exterior lighting 190. The electrical system is supported by heavy duty 12 gage wire feeding all the aforementioned elements protected in a control board with 5 15 amp pickups. The thermo cam will be regulated with a voltage variation transformer. Green color LED lighting for submersible outdoor use. Other components include 2×1 ptr rack with 200 liter container and space for hydro motor; Acii 240v model infrared motion sensor, thermo camera brand unit model uti 165h; 14 inch LCD monitor Seiki brand model sc-9ss 840n; and antibacterial gel dispenser brand Helvex model mb-1100.

Embodiments of the system of the present invention include a processor configured to control at least one process of the system and a memory for storing executable instructions which, when executed by the processor, cause the processor to provide an output to perform the at least one process. The processor provides output to perform the process of utilizing a thermo sensor for measuring and recording the body temperature of a person proximate the entrance of the system. The processor provides output to perform the process of utilizing a motion sensor for sensing motion of a person proximate the entrance of the system and activating the dispersal of the sanitizing solution via the nozzle. The processor provides output to perform the process of controlling the pressure of the pump delivering the sanitizing solution to the nozzle.

Referring to FIGS. 1, 8 and 9, a silhouette of a person P is shown near the entrance 132 of sanitizing nebulization system 100. Thermo sensor 160 and display 152 are located near entrance 132. The body temperature reading of person P as measured by thermo sensor 160 can be shown on display 152. The display can be a 20 inch screen. Thermo sensor 160 can be a thermo cam using infrared technology.

Also located near entrance 132 of sanitizing nebulization system 100 is a hand sanitizer gel dispenser 180. As person P enters system 100, motion sensor 170 detects the presence of the person about to enter the device and activates nozzles 140 to disperse the sanitizing solution in the form of a nebulized fog F to expose the person to the sanitizing solution as the person walks through system 100.

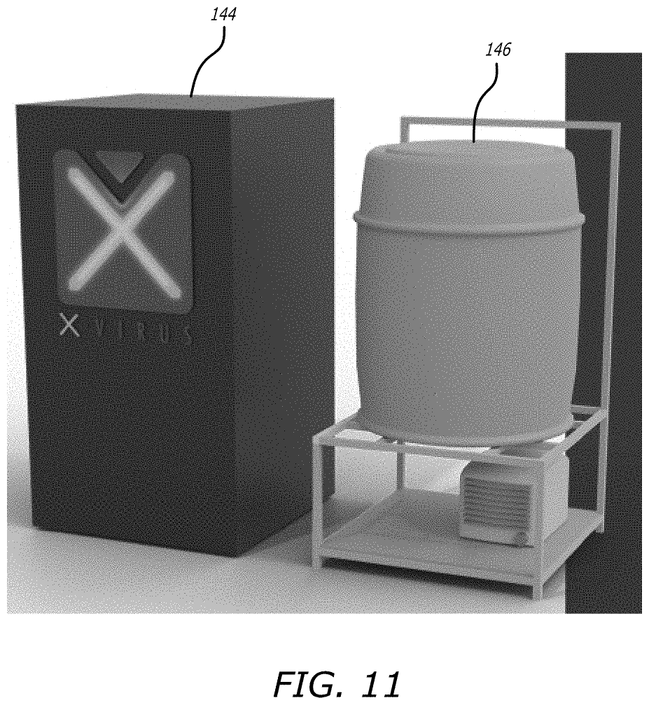

FIGS. 10A-10C show a side elevation, front end view, and front side perspective view, respectively of system 100. An embodiment of sanitizing system 100 has height of approximately 2.5 m, a width of approximately 1.5 m, and a length of approximately 2.5 m. As an example, an embodiment of the present invention includes a nebulization system with 10 nebulizers (nozzle) and a 200 liter container of sanitizing solution.

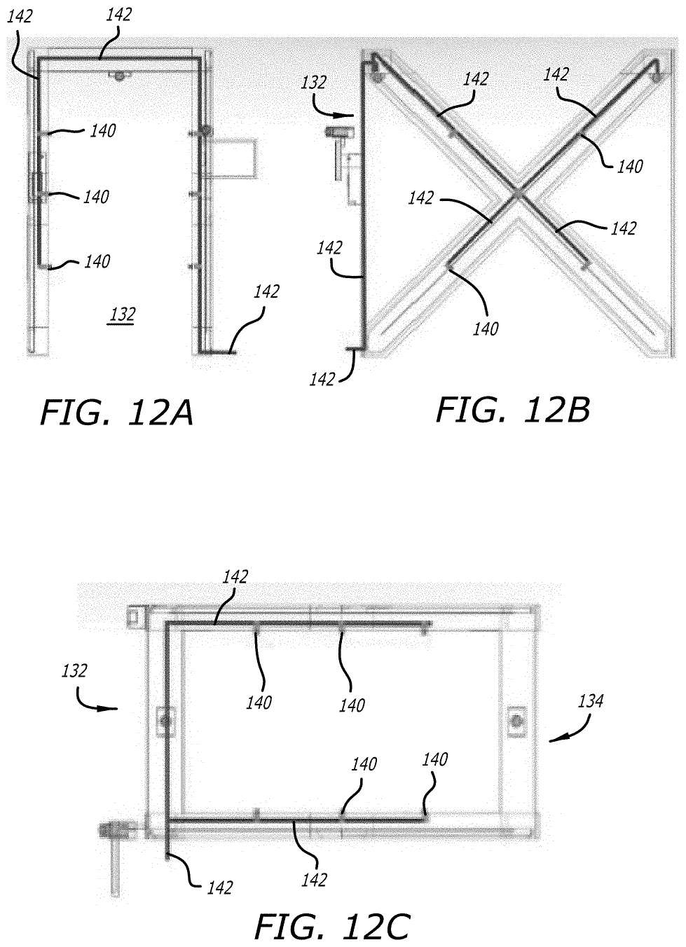

FIG. 11 is a front perspective view of an embodiment of sanitizing solution container 146, pump 144, and power supply of a preferred embodiment of sanitizing nebulization system 100. The power supply can be a wall outlet, a battery, and/or a generator.

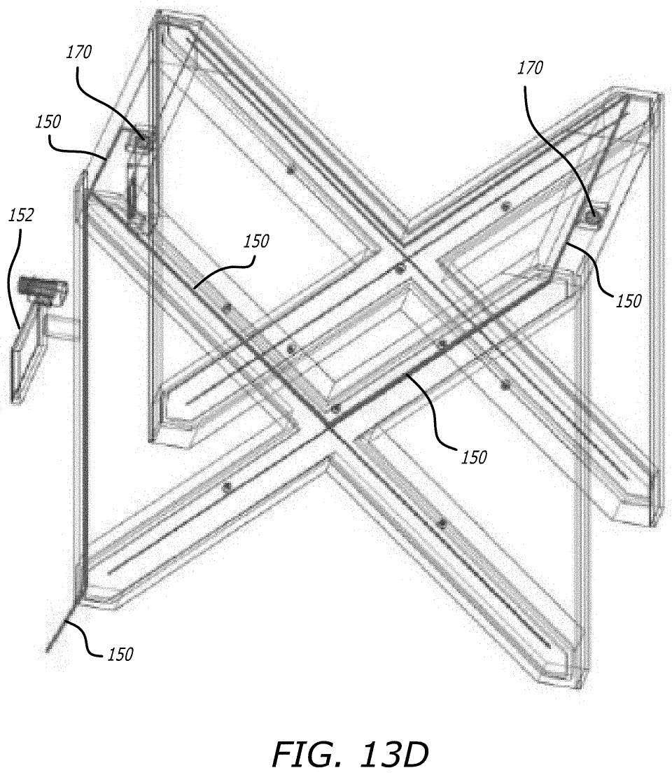

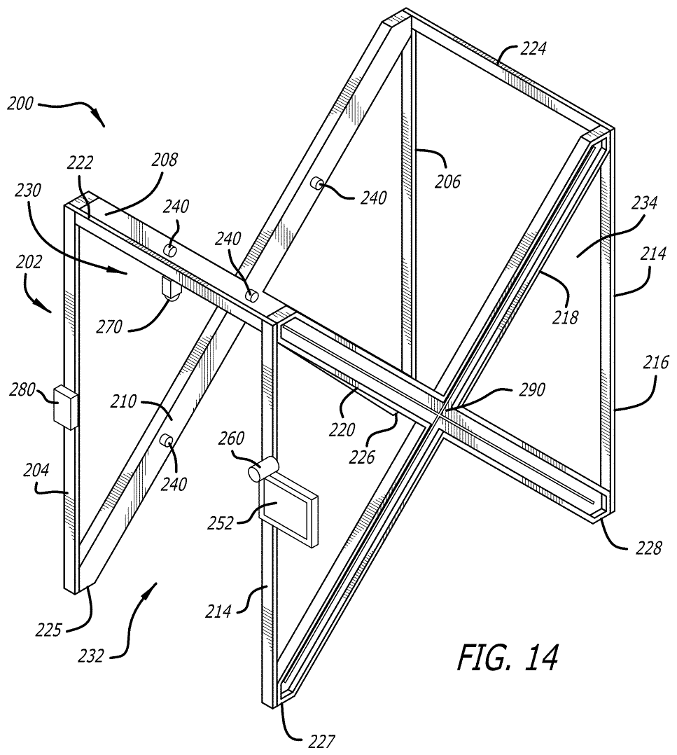

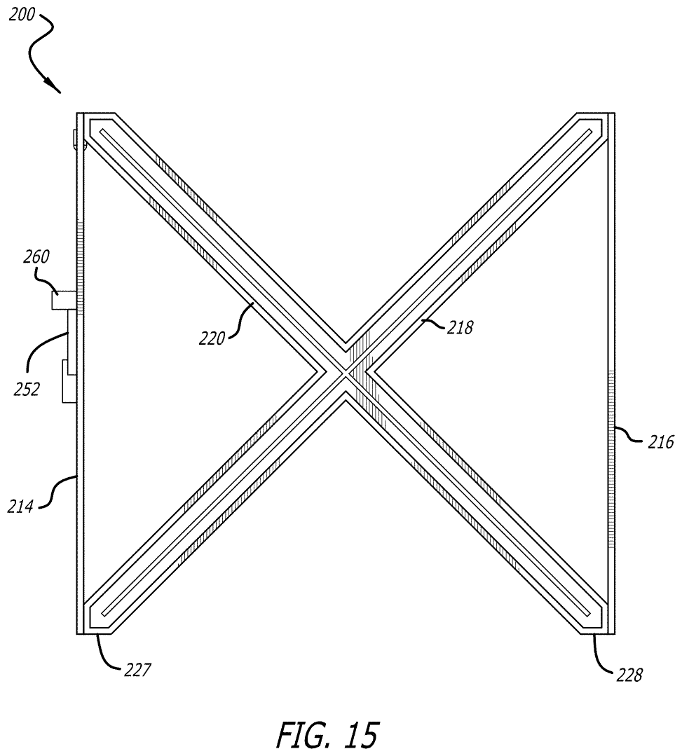

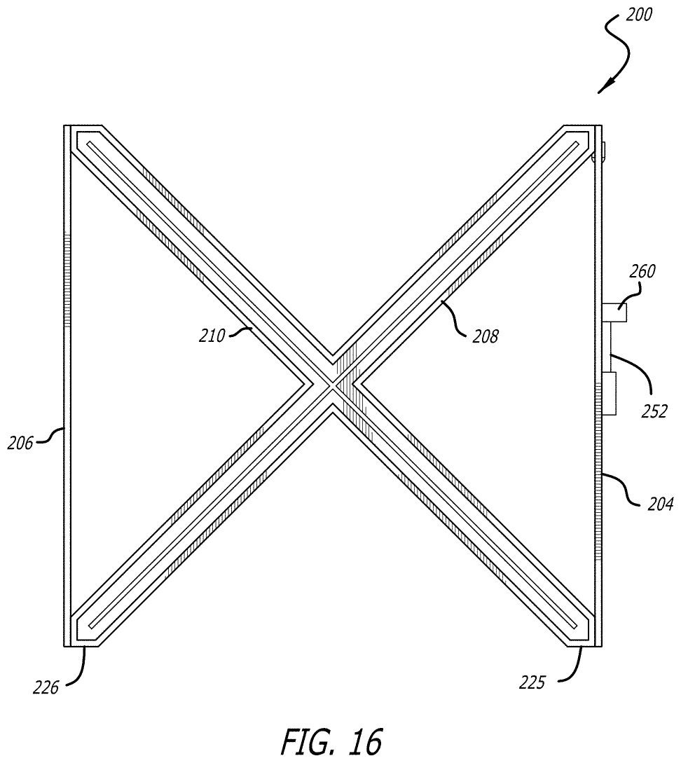

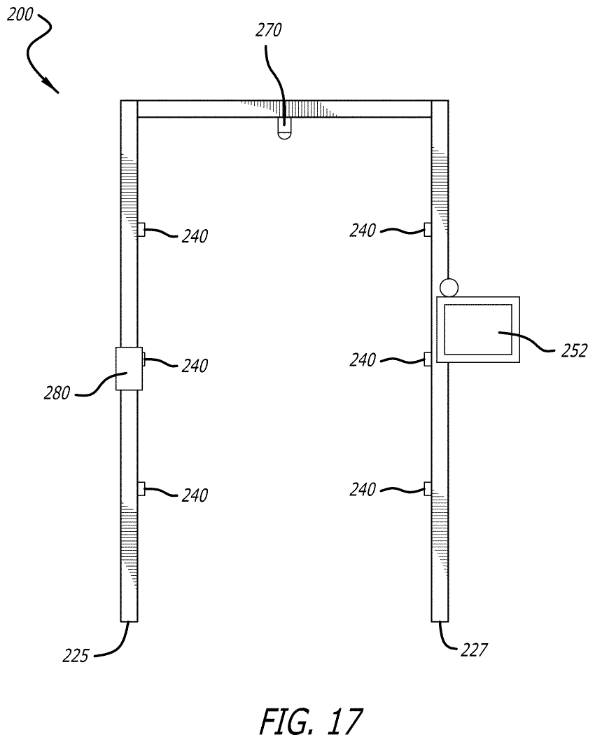

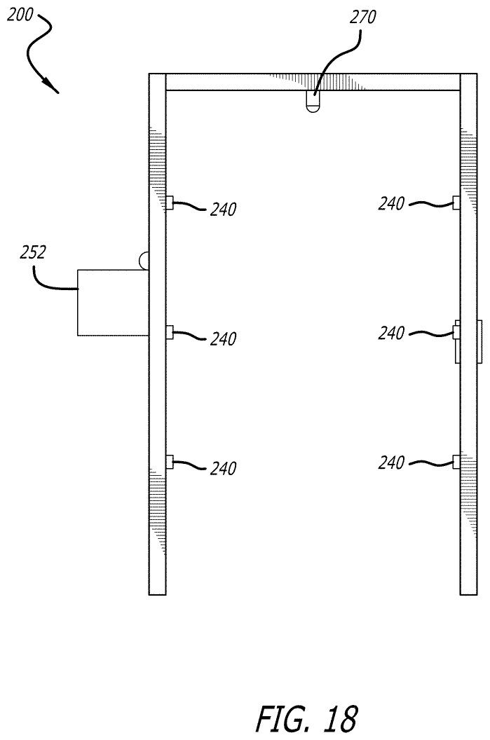

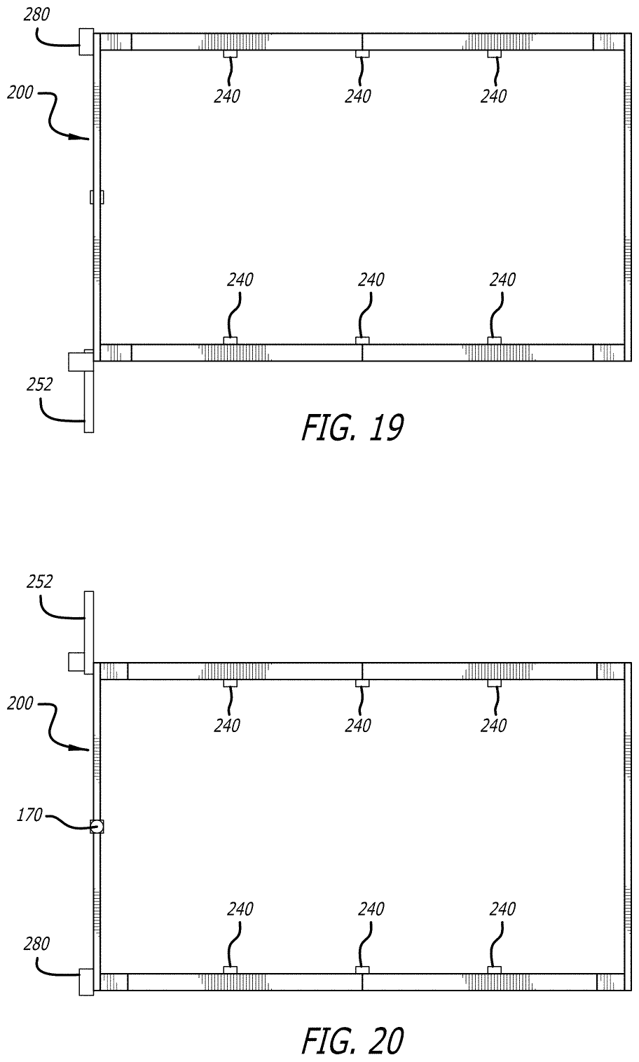

Referring to FIGS. 14-20, another embodiment of the sanitizing nebulization system of the present is shown. Sanitizing nebulization system 200 is similar to sanitization nebulization 100 described above. Accordingly, the description of sanitizing nebulization system 100 is incorporated by reference herein and reference numbers in a “200” series are used to identify like elements identified by corresponding reference numbers in the “100” series in the drawings.

Sanitizing nebulization system 200 has a thinner overall profile that is less intrusive and more open than system 100. The configuration of the system facilitates placement and transportation while reducing costs of manufacture and shipping.

The foregoing outlines features of several embodiments so that those of ordinary skill in the art may better understand various aspects of the present disclosure. Those of ordinary skill in the art should appreciate that they may readily use the present disclosure as a basis for designing or modifying other processes and structures for carrying out the same purposes and/or achieving the same advantages of various embodiments introduced herein. Those of ordinary skill in the art should also realize that such equivalent constructions do not depart from the spirit and scope of the present disclosure, and that they may make various changes, substitutions, and alterations herein without departing from the spirit and scope of the present disclosure.

Although the subject matter has been described in language specific to structural features or methodological acts, it is to be understood that the subject matter of the appended claims is not necessarily limited to the specific features or acts described above. Rather, the specific features and acts described above are disclosed as example forms of implementing at least some of the claims.

Various operations of embodiments are provided herein. The order in which some or all of the operations are described should not be construed to imply that these operations are necessarily order dependent. Alternative ordering will be appreciated having the benefit of this description. Further, it will be understood that not all operations are necessarily present in each embodiment provided herein. Also, it will be understood that not all operations are necessary in some embodiments.

Moreover, “exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application and the appended claims are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, or variants thereof are used, such terms are intended to be inclusive in a manner similar to the term “comprising”. Also, unless specified otherwise, “first,” “second,” or the like are not intended to imply a temporal aspect, a spatial aspect, an ordering, etc. Rather, such terms are merely used as identifiers, names, etc. for features, elements, items, etc. For example, a first element and a second element generally correspond to element A and element B or two different or two identical elements or the same element.

Also, although the disclosure has been shown and described with respect to one or more implementations, equivalent alterations and modifications will occur to others of ordinary skill in the art based upon a reading and understanding of this specification and the annexed drawings. The disclosure comprises all such modifications and alterations and is limited only by the scope of the following claims. In particular regard to the various functions performed by the above described features (e.g., elements, resources, etc.), the terms used to describe such features are intended to correspond, unless otherwise indicated, to any features which performs the specified function of the described features (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

Examples or embodiments of the subject matter and the functional operations described herein can be implemented in digital electronic circuitry, or in computer software, firmware, or hardware, including the structures disclosed in this specification and their structural equivalents, or in combinations of one or more of them.

Some examples or embodiments are implemented using one or more modules of computer program instructions encoded on a computer-readable medium for execution by, or to control the operation of, a data processing apparatus. The computer-readable medium can be a manufactured product, such as hard drive in a computer system or an embedded system. The computer-readable medium can be acquired separately and later encoded with the one or more modules of computer program instructions, such as by delivery of the one or more modules of computer program instructions over a wired or wireless network. The computer-readable medium can be a machine-readable storage device, a machine-readable storage substrate, a memory device, or a combination of one or more of them.

The terms “computing device” and “data processing apparatus” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment, or a combination of one or more of them. In addition, the apparatus can employ various different computing model infrastructures, such as web services, distributed computing and grid computing infrastructures.

The processes and logic flows described in this specification can be performed by one or more programmable processors executing one or more computer programs to perform functions by operating on input data and generating output.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A walkthrough sanitizing nebulization system, comprising:
an open frame structure having opposite side portions spaced apart to define an interior area therebetween, an open top area in communication with the interior area, an entrance at one end to access the interior area, and an exit at the opposite end of the interior area, the opposite side portions having an open configuration, the interior area being in open communication with the exterior environment surrounding the open frame structure through the open configuration of the opposite side portions, the open frame structure being sized and configured to allow a person to walk through by entering through the entrance and exiting through the exit;
at least one nozzle for dispersing a sanitizing solution to the interior area of the open frame structure, the nozzle configured to disperse the solution as at least one of a fog and mist to at least a portion of the interior area of the open frame structure;
a hydraulic system coupled to the nozzle for delivering the solution to the nozzle, the hydraulic system including a pump for delivering the solution from a storage container, the pump delivering the solution to the nozzle under pressure to generate the at least one of a fog and mist; and
an electrical system coupled to a power source and to the pump,
wherein when the person passes through the entrance and walks through the interior area of the open frame structure, the person is exposed to the at least one of fog and mist dispersal of the solution.

2. The system of claim 1, further comprising a motion sensor for activating the nozzle to disperse the solution.

3. The system of claim 1, further comprising a thermo sensor for measuring the body temperature of a person entering the open frame structure.

4. The system of claim 3, further comprising a display for displaying the body temperature of a person entering the structure.

5. The system of claim 1, further comprising a hand sanitizer dispenser.

6. The system of claim 1, further comprising multiple nozzles positioned along the side portions of the open frame structure and facing the interior area.

7. The system of claim 1, wherein the opposite side portions have top sides and are interconnected at least at one end to by an interconnecting member proximate the top sides.

8. The system of claim 1, wherein each of the opposite side portions have an X-shaped configuration.

9. The system of claim 1, wherein the open frame structure has a length of approximately 2.5 meters, a width of approximately 1.5 meters, and a height of approximately 2.5 meters.

10. The system of claim 1, further comprising a processor configured to control at least one process of the system and a memory, the memory storing executable instructions which, when executed by the processor, cause the processor to provide an output to perform the at least one process.

11. The system of claim 10, wherein the processor provides output to perform the process of utilizing a thermo sensor for measuring and recording the body temperature of a person proximate the entrance of the system.

12. The system of claim 10, wherein the processor provides output to perform the process of utilizing a motion sensor for sensing motion of a person proximate the entrance of the system and activating the dispersal of the sanitizing solution via the nozzle.

13. The system of claim 10, wherein the processor provides output to perform the process of controlling the pressure of the pump delivering the sanitizing solution to the nozzle.

14. A method for disinfecting people with a walk-through sanitizing nebulization system, comprising:
providing an open frame structure having:
opposite side portions spaced apart to define an interior area therebetween, an open top area in communication with the interior area, an entrance at one end to access the interior area, and an exit at the opposite end of the interior area, the opposite side portions having an open configuration, the interior area being in open communication with the exterior environment surrounding the open frame structure through the open configuration of the opposite side portions, the open frame structure being sized and configured to allow a person to walk through by entering through the entrance and exiting through the exit;
at least one nozzle for dispersing a sanitizing solution to the interior area of the open frame structure, the nozzle configured to disperse the solution as at least one of a fog and mist to at least a portion of the interior area of the open frame structure; and
a hydraulic system coupled to the nozzle for delivering the solution to the nozzle, the hydraulic system including a pump for delivering the solution from a storage container, the pump delivering the solution to the nozzle under pressure to generate the at least one of a fog and mist;
measuring body temperature of a person proximate the open frame structure;
displaying the body temperature of the person on a display; and
activating, with a motion sensor, the nozzle to disperse sanitizing solution to a person walking through the open frame structure.

15. The method of claim 14, further comprising dispensing sanitizing solution to at least 12 persons per minute walking through the frame structure.

16. The method of claim 14, further comprising dispensing sanitizing solution via multiple nozzles to a person walking through the frame structure
